(19) United States
(12) Patent Application Publication
(10) Pub. No.: US 2021/0178143 A1
(43) Pub. Date: Jun. 17, 2021

(54) NONDISCONNECTABLE, SECURABLE CONNECTOR HAVING A CAPPING SLEEVE

(71) Applicant: CODAN Holding GmbH, Lensahn (DE)

(72) Inventor: Jozef Jacobus Maria CROMBACH, Nieuwleusen (NL)

(73) Assignee: CODAN Holding GmbH, Lensahn (DE)

(21) Appl. No.: 16/715,105

(22) Filed: Dec. 16, 2019

(51) Int. Cl. A61M 39/10; A61M 39/12; A61M 39/20

(52) U.S. Cl. CPC A61M 39/1011; A61M 39/12; A61M 39/20; A61M 2039/1033; A61M 2039/1027; A61M 2039/1066; A61M 2039/1077

(57) ABSTRACT

The present invention relates to a connector for use in supply lines for intravenous infusions. The object of the present invention is to provide a connector designed in such a way that secure assembly of the infusion arrangement is made possible, and such that the connecting piece may be nondisconnectably or nonreleasably locked to the mating piece at a later time. This object is achieved by providing securing of the connection, in particular by way of a thread, and providing a separately operable capping sleeve that covers the securing.

### TECHNICAL FIELD

The present invention relates to a connector, in particular for use in supply lines for intravenous infusions, in particular a Luer lock system, that can be connected to a mating piece, the connector having a capping sleeve that can be locked on the connector, thus preventing/hindering disconnection of the connector.

### BACKGROUND

### Background Information

Connectors for connecting various sections of a system have been known for quite some time. In particular a form-fit connection of both sections is necessary to allow transport of a substance through the overall system without loss of the substance, while preventing penetration of foreign matter at the connection. In particular, such a system includes a connecting piece and a mating piece, in particular a positive connecting piece, and a negative connecting piece as a mating piece. A Luer lock system that allows transport of a liquid without loss at a connection is particularly advantageously used in a medical system. The connecting piece includes a cone, and in the Luer lock system contains a thread, which ensure a connection that is form-fit and tight as well as secure. In particular in the field of medical technology, various connectors have been known for quite some time; the Luer lock system has become prevalent and is predominantly used. The shapes of the connector are defined by the ISO 80369-7 and DIN EN 20594 standards, so that systems from different manufacturers are compatible and combinable with one another. The Luer system allows the connecting piece and the mating piece, in particular the positive connecting piece and the negative connecting piece, in particular the male connecting piece and the female mating piece, of a Luer system to be easily connected to one another. The connecting piece and the mating piece may have threads so that the connecting piece and the mating piece may be screwed together, one inside the other. This allows connection of two sections, so that the connecting piece and the mating piece cannot be inadvertently disconnected from one another. Such an arrangement is referred to as a Luer lock system. One disadvantage of such a system is that it is disconnectable. Such a disconnection may occur intentionally or unintentionally in the course of further handling during or in preparation of the system, in particular for a medical treatment.

Various securing mechanisms are known in the prior art for reliably avoiding disconnection. EP 1917996 B1, among other sources, discloses a Luer lock system that allows rotation of the thread only in the screwing-in direction, thus securing against disconnection. As soon as an appropriate connection is secured by the screwing-in, the screw connection can no longer be released manually. This is achieved in that the Luer lock has toothing situated around the connecting piece, and the lock thread, which is rotatable about the connecting piece, has correspondingly fitting countertoothing, so that in a locked state the capping sleeve nonreleasably connects the connecting piece to the mating piece by way of the toothing within the thread. This has the disadvantage that as soon as the infusion arrangement is assembled, this system is nonreleasably locked, so that subsequent disconnection is no longer possible.

One reason that releasing the connection might be necessary is an error in setting up the system, such as incorrect assembly of an infusion tree. In the use of known nondisconnectable connectors, this requires disposal of the faulty assembly, which in particular is costly and/or the only one available.

In contrast, the conventional Luer system (without a lock) allows correction, but does not secure against disconnection. Such an approach is also described in Utility Model DE 20109061 U1. The object of the cited document is to provide a connection that may be released with little effort. Such a system has a connecting piece for connection to a mating piece, and a coupling that is equipped with an overload coupling and that can be rotated onto the connecting piece, and when a limiting value of the torque is reached, the sleeve may be further rotated using a handle, but the connecting piece is not further entrained in the rotation. Overtightening the connection may be avoided in this way. This connection may be released by rotating in the opposite direction.

To ensure secure, fluid-tight retention of a hose on a connector, it has been proposed in EP 3081253 A1, for example, to provide protrusions on an end portion of the connector, which together with a nut, clamp the hose between the connector, the end portion of the connector, and the nut in such a way that secure retention of the hose on the connector results.

### SUMMARY

The object of the present invention is to provide a connector, in particular a Luer system, in particular a connector of a Luer lock system, that is designed in such a way that assembly of the infusion arrangement, in particular an infusion tree, with connection of at least one connecting piece to a mating piece, is made possible, and the connecting piece and the mating piece may be nondisconnectably and nonreleasably connected to one another or locked at a later time. A further object is that after assembly the infusion arrangement may be checked and the infusion arrangement may be disassembled, but the infusion arrangement is already ensured against inadvertent disconnection, and transferring the infusion arrangement into a nondisconnectable state cannot take place unintentionally. Further objects are to provide a corresponding system and method.

This object is achieved by a connector, in particular a medical connector, a system having multiple such connectors, and a method for producing an infusion arrangement, in particular an infusion tree. The connector and/or adapter has a connecting piece for connection to a fitting mating piece for the purpose of substance transport, wherein the connecting piece has a cone and a securing mechanism for reversibly securing a connection to the fitting mating piece, a hose, a displacement area, and a capping sleeve. The securing mechanism might be comprised of a thread, in particular a lock thread, in particular a Luer-Lock thread, of a member of the force-fitting type, a conical surface, in particular along the cone, in particular a conical Luer cone, any combination thereof or any other way of securing the connector with another part via the fitting mating piece of the connecting piece. The capping sleeve is configured to be at least partially rotatable about the connecting piece and/or on the displacement area. At least one locking member is provided on the connecting piece and/or on the capping sleeve for nonreleasably locking the connecting piece to the capping sleeve. The at least one locking member might be comprised of at least one bar, ring, partial or half ring, protrusion, indentation, thread, snap, groove, notch, slot, a tongue, any combination thereof or any other way of locking the capping sleeve with the connecting piece. The connector is designed in such a way that in the state in which the connecting piece is nonreleasably locked to the capping sleeve, displacement of the capping sleeve relative to the connecting piece is limited in all directions. The connector is characterized in that the capping sleeve and the displacement area are designed in such a way that the capping sleeve has at least one engagement mechanism that is configured so as to be at least partially connected to the displacement area, and thus to at least partially enclose the displacement area in such a way that between the at least one engagement mechanism and the displacement area, a resistance is present that acts against a displacement of the capping sleeve relative to the connecting piece, so that the force that is necessary to move the capping sleeve is greater than the weight force of the capping sleeve, and/or the engagement mechanism form an opening width that is selected so that the engagement mechanism rest on the displacement area, exerting a force compressing the displacement area with an undersize on the circumference of the displacement area, and/or the engagement mechanism define an opening that is smaller than a maximum extension of the cross section of the displacement area perpendicular to a longitudinal extension of the displacement area, and/or the capping sleeve is displaceable on the displacement area by means of a force of less than 5 N and/or greater than 0.01 N that acts parallel to the longitudinal extension of the displacement area and/or the capping sleeve, in particular in addition to the weight force of the capping sleeve. The at least one engagement mechanism might be comprised of at least a bar, a ring, a half or partial ring, any combination thereof or any other way of an engagement of the capping sleeve with the displacement area. The capping sleeve is designed in such a way that in the state in which the connecting piece is nonreleasably locked to the capping sleeve, the securing mechanism and all parts torsionally rigidly connected thereto are at least partially enclosed and/or covered, in particular by the capping sleeve, and/or access to portions of the connector that are usable for releasing the securing mechanism and/or for reversing the securing are at least partially or completely enclosed and/or covered, in particular by the capping sleeve, and/or operating and/or releasing the securing mechanism are/is hindered and/or prevented by at least partially covering the securing mechanism and/or the mating piece and/or the connector, in particular by covering by the capping sleeve.

The system has at least one connector that has a connecting piece and at least one mating piece.

The method for producing an infusion arrangement, in particular for producing an infusion tree for transport, with connection of the aforementioned connecting piece to a mating piece, and/or a connector includes reversibly connecting the connecting piece to the mating piece, and the connection is reversibly secured; the release of the reversible securing the release of the reversible securing is hindered and/or prevented by slipping on the capping sleeve and nonreleasably locking the capping sleeve to the connecting piece. The hindrance and/or prevention takes place by at least partially covering and/or at least partially enclosing the securing mechanism and/or the connecting piece and/or the mating piece used for the reversible securing, and/or by hindering and/or preventing access to the securing mechanism used for the reversible securing, and/or by hindering and/or preventing operation and/or release of the securing mechanism used for the reversible securing. The slipping on and the nonreleasable locking are accomplished by moving the capping sleeve on the connecting piece and/or a displacement area and/or a hose and/or tube adjoining same, and/or by moving the capping sleeve on the connecting piece and/or by slipping a capping sleeve onto the connecting piece.

Advantageous embodiments and refinements include that the capping sleeve is rotatable about the connector, at least in the state in which the capping sleeve is nonreleasably locked to the connecting piece. The at least one locking member is configured in such a way that the nonreleasable locking of the locking member requires a force of at least 27.5 N and/or at most 60 N. In the nonreleasably locked state of the connector, the connector is releasable only by at least partially destroying the capping sleeve and/or the connector. Releasing the reversible securing is hindered, in particular prevented, by the at least partial enclosure of the securing mechanism and/or connector and/or mating piece brought about by the capping sleeve in the nonreleasably locked state. The opening width formed by the engagement mechanism has a smaller diameter than the maximum outer diameter of the displacement area. The capping sleeve has at least one first locking member that forms a locking member opening width, and the connecting piece has at least one second locking member that forms a locking member outer diameter, wherein the diameter of the locking member opening width is less than 8 mm and/or is smaller than the locking member outer diameter, and/or the locking member of the connecting piece with their outer extensions define a circle having an outer diameter that is smaller than the inner diameter of a portion of the capping sleeve, in and/or on which the locking member of the connecting piece are accommodated and/or rest in the state in which the connecting piece is nonreleasably locked to the capping sleeve. The engagement mechanism has at least one bar.

Further advantageous embodiments and refinements include that the locking member are formed in such a way that the locking member have a trapezoidal shape in their cross-sectional plane parallel to the longitudinal extension of the connector and/or of the capping sleeve. The locking member of the capping sleeve and/or of the connecting piece, in particular in the state in which the connecting piece is locked to the capping sleeve, form a ring that is almost completely closed. The locking member of the connecting piece and/or of the capping sleeve, in a top view of the locking member parallel to the longitudinal extension of the connecting piece and/or of the capping sleeve, in each case form a segment of a ring, wherein the longest chord through each segment of the locking member of the connecting piece is longer than the distance between the locking member of the capping sleeve, which the locking member of the capping sleeve have in a top view of the locking member parallel to the longitudinal extension of the connecting piece and/or of the capping sleeve, and/or the locking member of the connecting piece and/or of the capping sleeve, in a top view of the locking member parallel to the longitudinal extension of the connecting piece and/or of the capping sleeve, in each case form a segment of a ring, wherein the longest chord through each segment of the locking member of the connecting piece is longer than the distance between the locking member of the connecting piece, which the locking member of the connecting piece have in a top view of the locking member parallel to the longitudinal extension of the connecting piece and/or of the capping sleeve and/or the locking member of the connecting piece and/or of the capping sleeve, in a top view of the locking member parallel to the longitudinal extension of the connecting piece and/or of the capping sleeve, in each case form a segment of a ring, wherein a first locking member of the connecting piece is at least partially covered by a locking member of the capping sleeve, and/or the locking member of the connecting piece and of the capping sleeve, in a top view of the locking member parallel to the longitudinal extension of the connecting piece and/or of the capping sleeve, in each case form a segment of a ring, wherein in the state in which the connecting piece is locked to the capping sleeve, in any possible position of the locking member of the connecting piece and of the capping sleeve, at least one locking member of the connecting piece is at least partially covered in each case by at least one locking member of the capping sleeve.

Further advantageous embodiments and refinements include that the securing mechanism is fixedly and/or rigidly connected to the cone and/or to the connecting piece. The engagement mechanism of the capping sleeve are provided distally on the capping sleeve and/or on the end of the capping sleeve remote from the securing mechanism, and/or the capping sleeve is positioned in such a way that the engagement mechanism of the capping sleeve are situated on the end of the connecting piece remote from the cone, and/or on the displacement area, on the end of the displacement area remote from the cone.

According to the invention, the object is achieved by a connector and/or adapter, in particular a Luer system, in particular a connector of a Luer lock system, wherein the connector has a connecting piece, in particular a positive connecting piece, that can be connected to a fitting mating piece, in particular a negative mating piece, in particular a standard negative Luer lock mating piece, for the purpose of transporting substances, in particular liquids. The connecting piece has a cone, in particular a Luer cone, and a securing mechanism, in particular a thread, in particular a lock thread, in particular a securing mechanism that at least partially encloses the cone, in particular a thread, for reversibly securing a connection of the connecting piece and the mating piece, in particular with interaction with at least one fitting and/or complementary countersecuring mechanism of the mating piece, for example a thread, in particular securing against inadvertent release of the connection. Furthermore, the connecting piece has a hose, which in particular is situated on the end, in particular on the end facing away from the cone, and/or which is connected to the cone, for transporting substances, in particular liquids. In addition, the connector has a displacement area, in particular the displacement area at least partially including the hose or a portion of the hose and/or at least a portion of the connecting piece, and/or the displacement area being formed on the hose and/or at least a portion of the connecting piece. The connector also has a capping sleeve, wherein the connector is designed in such a way that the capping sleeve in particular is situated so as to be displaceable on the displacement area, in particular on the hose, and/or is designed in such a way that the capping sleeve is situated so as to be at least partially rotatable about the connecting piece and/or the hose and/or the displacement area and/or the hose, in particular about the securing mechanism, and/or on the displacement area, wherein the rotatability is provided in particular about the longitudinal extension of the displacement area and/or about the axis of symmetry and/or longitudinal extension of the cone, in particular the Luer cone. In addition, at least one locking member is provided on the connector, in particular on the connecting piece, in particular on an end portion of the connecting piece situated opposite from the cone and/or an end portion of the displacement area facing the cone, and/or on the capping sleeve, to allow nonreleasable locking of the connecting piece to the capping sleeve, wherein the connector is designed in such a way that in the irreversibly locked state of the connector, in particular of the connecting piece to the capping sleeve, displacement of the capping sleeve relative to the connecting piece is limited in all directions.

In particular, at least one locking member is provided in each case on the connecting piece and on the capping sleeve, wherein the locking member are designed so that the locking member of the connecting piece can interact with the locking member of the capping sleeve in such a way that they can bring about the locking of the connecting piece to the capping sleeve. In particular, the at least one locking member is designed in such a way that displacement of the capping sleeve in the direction of the cone beyond a predefined position, in particular displacement to an end of the displacement area on the cone side, brings about the locking.

The capping sleeve and the displacement area are designed in such a way that the capping sleeve has at least one engagement mechanism, wherein the at least one engagement mechanism is configured so as to be at least partially connected to the displacement area, in particular connected with a force fit, and/or is configured so as to at least partially enclose the displacement area, in particular from at least two sides. The engagement mechanism and/or a portion of the, or the entire, displacement area may be designed in such a way that between the at least one engagement mechanism and the displacement area a resistance is present that acts against a displacement of the capping sleeve relative to the connecting piece, so that the force that is necessary to move the capping sleeve, in particular the engagement mechanism and/or the locking member, on the displacement area, in particular with respect to their/its relative position on the displacement area, in particular along the displacement area, is greater than the weight force of the capping sleeve, and in particular the force is at least 1.1 times the weight force of the capping sleeve, and in particular is greater than several times the weight force of the capping sleeve. Alternatively or additionally, the engagement mechanism and/or a portion of the, or the entire, displacement area may be designed in such a way that the engagement mechanism form an opening width that is selected so that the engagement mechanism rest on a portion of the, or the entire, displacement area, exerting a force compressing the displacement area with an undersize on the circumference of the displacement area. Alternatively or additionally, the engagement mechanism may be designed in such a way that the engagement mechanism define an opening that is smaller than a maximum extension of the cross section of a portion of the, or the entire, displacement area perpendicular to a longitudinal extension of the displacement area. Alternatively or additionally, the engagement mechanism and/or a portion of the, or the entire, displacement area may be designed in such a way that the capping sleeve is displaceable on the displacement area by means of a force of less than 5 N and/or greater than 0.01 N, in particular less than 1 N, in particular greater than 0.1 N, in particular with a force between 0.1 and 0.9 N, in particular between 0.12 and 0.81 N, that acts parallel to the longitudinal extension of the displacement area and/or the capping sleeve, in particular in addition to the weight force of the capping sleeve.

Due to such designs of the capping sleeve and/or of the displacement area, the capping sleeve is not moved from a neutral position on the displacement area without further action, such as an additional external force. An infusion system may thus be assembled, or the connector may be connected to a mating piece and secured by the securing mechanism, without the risk of the capping sleeves unintentionally being moved and/or locked. Corrections are thus possible until a nondisconnectable state is established by intentionally locking the capping sleeve.

The capping sleeve and/or the connecting piece, the connector, and/or the mating piece are/is designed in such a way that in the state in which the capping sleeve is nonreleasably locked to the connecting piece, in particular via the locking member, at least a portion of the connecting piece, the securing mechanism, and all parts rigidly connected thereto, in particular the connecting piece and/or the securing mechanism and/or the mating piece, in particular countersecuring mechanism of the mating piece, or all of their accessible areas, are at least partially or completely enclosed and/or covered, in particular by the capping sleeve, and/or in the state in which the connecting piece is nonreleasably locked to the capping sleeve, access to portions of the connector, in particular of the securing mechanism, and/or the mating piece, in particular the countersecuring mechanism, in particular the positive connecting piece, and/or of the mating piece, that are usable for releasing the securing mechanism and/or countersecuring mechanism and/or for reversing the securing, in particular between the connecting piece and the mating piece, or all of their accessible areas, are at least partially or completely enclosed and/or covered, in particular by the capping sleeve, and/or in the state in which the connecting piece is nonreleasably locked to the capping sleeve, operating and/or releasing the securing mechanism and/or countersecuring mechanism, and/or reversing the securing by at least partially or completely covering and/or enclosing the securing mechanism and/or connecting piece and/or countersecuring mechanism and/or the mating piece and/or the connector and/or all of their accessible areas, are/is hindered and/or prevented, in particular by covering and/or enclosure by the capping sleeve.

The connector is advantageously designed in such a way that at least in the state in which the capping sleeve is nonreleasably locked and/or not nonreleasably locked to the connecting piece, the capping sleeve is rotatable about a rotational axis about the connector, in particular about the connecting piece and/or the securing mechanism and/or the hose and/or the displacement area, in particular in all rotation directions, wherein in particular the torque necessary for the rotation is less than 0.08 Nm, in particular less than 0.01 Nm, and in particular the capping sleeve is freely rotatable.

The displacement area advantageously has a longitudinal extension, in particular a longitudinal extension parallel to the longitudinal extension of the connecting piece and/or the capping sleeve, of at least 1 cm, in particular at least 5 cm, in particular at least 10 cm.

The engagement mechanism of the capping sleeve is advantageously configured in such a way that the resistance acting against a displacement of the capping sleeve relative to the connecting piece is present on the displacement area for a length of at least 1 cm, in particular at least 5 cm, in particular at least 10 cm, and/or on at least 10%, in particular at least 50%, of the length of the displacement area.

The capping sleeve is in particular rotatable about a rotational axis in two opposite rotation directions, wherein the rotational axis is in particular parallel to the longitudinal extension of the displacement area and/or of the capping sleeve and/or of the connector and/or of the longitudinal extension of the cone, in particular the Luer cone. The rotational axis represents a rotational axis of symmetry, in particular at least for a portion of the connector and/or the hose.

The at least one locking member is advantageously configured in such a way that the irreversible and/or nonreleasable locking of the locking member requires a force of at least 27.5 N and/or at most 60 N, in particular at least 35 N and/or at most 55 N, in particular at least 30 N and/or at most 51 N.

In the nonreleasably locked state of the connector, the connector is advantageously releasable only by at least partially destroying and/or partially deforming the connector and/or mating piece, in particular the capping sleeve, in particular by partially destroying the capping sleeve and/or the connecting piece.

The release and/or reversal of the reversible securing, in particular loosening a screw connection, between the connector, in particular the connecting piece, and the mating piece, in the nonreleasably locked state is advantageously hindered in particular by the capping sleeve in the nonreleasably locked state at least partially or completely enclosing the securing mechanism and/or countersecuring mechanism and in particular all parts torsionally rigidly connected thereto, and/or the connector and/or the mating piece, in particular all parts torsionally rigidly connected thereto.

The opening width formed by the engagement mechanism of the capping sleeve of the connector advantageously has a smaller diameter than the maximum outer diameter of the displacement area and/or of the, or a portion of the, displacement area. Such an opening width particularly advantageously has a diameter that is at least 0.05 mm and/or at most 0.1 mm smaller than the maximum outer diameter of the displacement area and/or of the, or a portion of the, displacement area, and/or a diameter that is least 5% smaller than the maximum outer diameter of the displacement area and/or of the, or a portion of the, displacement area.

The capping sleeve of the connector advantageously has at least one first locking member that forms a locking member opening width, and the connecting piece has at least one second locking member that defines a locking member outer diameter, wherein the diameter of the locking member opening width is less than 8 mm, in particular at least 5.5 mm, in particular 6.1±0.5 mm, and/or smaller, in particular at least 2% smaller, than the locking member outer diameter defined by the locking member of the connecting piece. Additionally or alternatively, the locking member of the connecting piece with their outer extensions define an outer diameter that is smaller, in particular 2% smaller, than the inner diameter of a portion of the capping sleeve, in and/or on which the locking member of the connecting piece are accommodated and/or rest in the state in which the connecting piece is nonreleasably locked to the capping sleeve, and in particular has an outer diameter of at most 8 mm and/or an outer diameter that is larger, in particular at least 2% larger, than the diameter of the opening width formed by the engagement mechanism, in particular an outer diameter of at least 5.5 mm, in particular 6.6±0.1 mm.

The connector and in particular the at least one locking member, in particular first and second locking member, are advantageously designed in such a way that in particular with application of force and reversible deformation of the locking member, in particular the first and/or second locking member, they are movable over one another in a movement direction, in particular along the longitudinal extension of the connector, of the connecting piece, of the displacement area, of the capping sleeve, and/or of the hose, and/or in a second movement direction opposite from the first movement direction are movable over one another only by destroying and/or inelastiscally deforming the at least one locking member.

The engagement mechanism of the capping sleeve of the connector advantageously have at least one bar, in particular at least two oppositely situated bars, in particular at least three bars, in particular more than three bars, that form circular segments having equal widths and/or having equal distances from one another.

The locking member of the capping sleeve and/or of the connecting piece in each case advantageously have at least one bar, in particular a locking bar, in each case in particular at least two oppositely situated bars, in particular locking bars, in each case in particular at least three bars, in particular locking bars, that form ring segments, in particular having equal widths and/or having equal distances from one another. The ring segments in each case extend in particular over 10 to 30°, in particular 15 to 25°, in particular 20°, of a circle about the midpoint.

The locking member of the capping sleeve and/or of the connecting piece are advantageously designed in such a way that they have a trapezoidal shape in their cross-sectional plane parallel to the longitudinal extension of the connector and/or of the capping sleeve, wherein this trapezoidal shape in particular has an interior angle greater than 40° and less than 50°, in particular 45°, wherein this trapezoidal shape in particular has an interior angle, perpendicular to the longitudinal extension of the capping sleeve and parallel to the longitudinal extension of the capping sleeve, greater than 40° and less than 50°, in particular 45°.

The locking member of the capping sleeve and/or of the connecting piece, in particular in the state in which the connecting piece is nonreleasably locked to the capping sleeve, in particular in a top view of the locking member parallel to the longitudinal extension of the connecting piece and/or of the capping sleeve, in particular together, advantageously form a ring that is almost completely, in particular at least 90%, closed. In particular, the locking member form such a ring regardless of the rotational position of the capping sleeve, i.e., in particular regardless of a rotation about the rotational axis.

The locking member of the connecting piece, in a top view of the locking member parallel to the longitudinal extension of the connecting piece and/or of the capping sleeve, in each case advantageously form a segment of a ring, wherein the longest chord through each segment of the locking member of the connecting piece is longer than the distance between the locking member of the capping sleeve, in particular the adjacent locking member of the capping sleeve, in particular adjacent locking member of the capping sleeve, which the locking member of the capping sleeve have in a top view of the locking member parallel to the longitudinal extension of the connecting piece and/or of the capping sleeve, and/or wherein the central angle of the connecting piece, over which the ring segment extends, is greater than the central angle between the locking member of the capping sleeve, in particular the adjacent locking member of the capping sleeve, in particular adjacent locking member of the connecting piece. Additionally and/or alternatively, the locking member of the capping sleeve may be designed in such a way that, in a top view of the locking member parallel to the longitudinal extension of the connecting piece and/or of the capping sleeve, in each case they form a segment of a ring, wherein the longest chord through each segment of the locking member of the capping sleeve is longer than the distance between the locking member of the connecting piece, and/or wherein the central angle segment of the capping sleeve over which the ring extends is greater than the central angle between the locking member of the connecting piece, in particular the adjacent locking member of the connecting piece, in particular of the successive locking member of the connecting piece in a ring, which the locking member of the connecting piece have in a top view of the locking member parallel to the longitudinal extension of the connecting piece and/or of the capping sleeve. Additionally and/or alternatively, the locking member of the connecting piece and/or of the capping sleeve may be designed in such a way that in a top view of the locking member parallel to the longitudinal extension of the connecting piece and/or of the capping sleeve, in each case they form a segment of ring, wherein a first locking member of the connecting piece is at least partially covered by a second locking member of the capping sleeve, and in particular each locking member of the connecting piece is simultaneously at least partially covered in each case by at least one, in particular two, locking member of the capping sleeve, wherein in particular such covering takes place regardless of the rotational position of the capping sleeve relative to the connecting piece. Additionally and/or alternatively, the locking member of the connecting piece and of the capping sleeve may be designed in such a way that in a top view of the locking member parallel to the longitudinal extension of the connecting piece and/or of the capping sleeve, in each case they form a segment of a ring, wherein in the state in which the connecting piece is nonreleasably locked to the capping sleeve, in each rotational position of the locking member of the connecting piece and of the capping sleeve, at least one locking member of the connecting piece is at least partially covered in each case by at least one, in particular two, locking member of the capping sleeve.

As the result of such designs of the locking member, an arrangement may be achieved which allows particularly simple, secure locking that is not susceptible to errors.

The securing mechanism, in particular the thread, in particular the lock thread, of the connecting piece of the connector is advantageously fixedly and/or rigidly connected to the cone, in particular the Luer cone, and/or to the connecting piece, preferably formed in one piece with same.

The engagement mechanism of the capping sleeve are advantageously provided distally on the capping sleeve and/or on the end of the capping sleeve remote from the securing mechanism, and/or the engagement mechanism of the capping sleeve are situated on the end of the connecting piece and/or of the displacement area remote from the cone, in particular on the hose.

A particularly compact and easily operable design is thus achieved.

The connector advantageously has a spacer piece that is provided between the securing mechanism, in particular the thread, in particular the Luer thread, and an end-position portion of the connecting piece, in particular the portion of the connecting piece opposite from the securing mechanism, wherein the outer circumference of a first portion, in particular a portion parallel to the longitudinal extension of the connector, of the spacer piece in areas has a larger circumference than the outer circumference of a second portion of the spacer piece, in particular a circumference whose diameter is greater than the circumference that forms the displacement area, and in particular is less than the diameter of the capping sleeve at a portion having the largest inner circumference. In particular, the spacer piece is only partially provided in a longitudinal extension parallel to the longitudinal extension of the cone, in particular the Luer cone. In this way, the screw connection of the mating piece with the thread that is torsionally rigidly connected to the spacer piece and all parts of the connecting piece torsionally rigidly connected thereto may be established in a particularly advantageous manner. A handle that forms this spacer piece is advantageously covered by the connecting piece when the capping sleeve is pulled over and the capping sleeve is locked.

In particular, the connector is designed with a hose that adjoins the connecting piece and that in particular has an outer diameter of 3.95 to 4.15 mm. In particular, the capping sleeve has a length of 20 to 40 mm, and/or the displacement area has a length of at least 20 to 40 mm and/or at least the length of the capping sleeve. In particular, the capping sleeve has an outer diameter of 8 to 20 mm. In particular, the connecting piece has a length of 20 to 40 mm, and/or the capping sleeve has a length of at least 80%, in particular at least 90%, of the length of the connecting piece. In particular, the connecting piece and/or the capping sleeve are/is designed in such a way that in the nonreleasably locked state, the relative movement between the connecting piece and the capping sleeve in the direction of its longitudinal extension and/or in the direction of the displacement on the displacement area is at most 4 mm, in particular at most 2 mm.

In particular, the distance between the engagement mechanism and the locking member of the capping sleeve is 0.5 to 3 mm, in particular 0.8 to 2 mm.

The object is further achieved by a system having at least one connector according to the invention and at least one mating piece, wherein the mating piece and/or the connector are/is configured to be connected for the purpose of substance transport, in particular liquid transport, and/or wherein the mating piece in particular is a negative mating piece, in particular a standard negative Luer lock mating piece.

In particular, the mating piece has a countersecuring mechanism, in particular a mating thread that fits the thread. Further advantageous embodiments described above are applicable to the system.

The object is further achieved by a method for producing an infusion arrangement, in particular for transporting and/or for administering substances, in particular liquids, in particular of an infusion tree, wherein this takes place with and/or by connection of a connecting piece, in particular a connector according to the invention, to a mating piece, in particular using a system and/or connector according to the invention, wherein the connecting piece is reversibly connected to the mating piece, and this connection is reversibly secured, in particular by securing mechanism, in particular by a reversible screw connection, and the release of the reversible securing is hindered and/or prevented by slipping on, in particular pulling over, a capping sleeve and nonreleasably locking the capping sleeve to the connecting piece, wherein the hindrance and/or prevention take(s) place by at least partially covering and/or at least partially enclosing the securing mechanism and/or the connecting piece and/or the mating piece used for the reversible securing, and/or by hindering and/or preventing access to the securing mechanism used for the reversible securing, and/or by hindering and/or preventing operation and/or release of the securing mechanism used for the reversible securing, wherein the hindrance and/or prevention are/is brought about in particular by slipping on the capping sleeve, and/or the position and/or configuration of the capping sleeve thus achieved, in particular relative to the connecting piece, mating piece, and/or securing mechanism.

The slipping on and the nonreleasable locking take place by moving the capping sleeve on the connecting piece and/or a displacement area and/or a hose and/or tube adjoining same, and/or by moving the capping sleeve on the connecting piece and/or by slipping a capping sleeve onto the connecting piece.

By slipping the capping sleeve in particular on and/or onto the connecting piece, and nonreleasably locking the capping sleeve to the connecting piece, releasing the reversible securing is hindered and/or prevented, and the reversibly secured connection is thus changed into a nondisconnectable connection.

The slipping on advantageously takes place by moving, in particular displacing, the capping sleeve on and/or along a/the displacement area, wherein the displacement area has a longitudinal extension, in particular a longitudinal extension parallel to the longitudinal extension of the connecting piece and/or capping sleeve, of at least 1 cm, in particular at least 5 cm, in particular at least 10 cm, and/or the slipping on takes place by moving, in particular displacing, the capping sleeve by at least 1 cm, in particular at least 5 cm, in particular at least 10 cm, on and/or along a/the displacement area, wherein in particular an additional force and/or a resistance acting against a displacement of the capping sleeve relative to the connecting piece must be overcome during the slipping on.

Such a length of the displacement area or slipping on the capping sleeve ensures that the arrangement or the connector may be easily assembled, and in particular the capping sleeve may be slipped onto the connector from an area that is remote from the connector, and that the arrangement or the connector may be easily assembled prior to the slipping on.

The resistance acting against a displacement of the capping sleeve relative to the connecting piece is advantageously present on the displacement area for a length of at least 1 cm, in particular at least 5 cm, in particular at least 10 cm, and/or on at least 10%, in particular at least 50%, of the length of the displacement area.

The capping sleeve is advantageously initially situated on the hose and/or the tube adjoining the connecting piece, and the capping sleeve is moved, in particular relative to the connecting piece, on the hose and/or the tube and/or on a portion of the connecting piece and/or the displacement area by means of a force, in particular an external force, in particular applied by a human hand, in particular applied to the capping sleeve, in particular a force parallel to the longitudinal extension of the capping sleeve, in particular in addition to the weight force of the capping sleeve. In particular, the force thereby applied is greater than the weight force of the capping sleeve, and in particular the force is at least 1.1 times the weight force of the capping sleeve, and in particular is greater than several times the weight force of the capping sleeve. The capping sleeve is advantageously moved due to an applied force of less than 5 N and/or greater than 0.01 N, in particular less than 1 N, in particular greater than 0.1 N, in particular between 0.12 and 0.81 N. In particular a force of at most 60 N and/or at least 27.5 N, in particular 35-55 N, in particular 36-52 N, is applied in order to lock the capping sleeve to the connecting piece. A displacement of the capping sleeve relative to the connecting piece is limited in all directions in particular due to the locking, in particular via an appropriate design of the connector.

Due to such an additional necessary and/or applied force for moving the capping sleeve on the displacement area in order to lock the capping sleeve to the connecting piece, unintentionally locking the capping sleeve to the connecting piece is prevented in a particularly advantageous manner, wherein during the assembly of the infusion arrangement and after a check of the arrangement by locking the capping sleeve to the connecting piece, release of the reversible securing between the connecting piece and the mating piece is prevented or hindered.

The above statements with regard to the properties of the connection, the nonreleasable locking, and the connector and the mating piece, advantageously apply to the system and/or the connector. This may be implemented according to the method in particular by an appropriate design and/or selection of the connecting piece, mating piece, securing mechanism, locking member, and/or connector used.

### BRIEF DESCRIPTION OF THE SEVERAL VIEWS OF THE DRAWINGS

Further advantageous features are explained below, strictly by way of example and in a nonlimiting manner, with reference to the purely schematic figures, which show the following:

FIG. 1: shows a longitudinal section of a connector with a connecting piece according to the invention, with an unlocked capping sleeve that is displaceably situated on the hose;

FIG. 2: shows a longitudinal section of the connector from FIG. 1 in the state in which it is connected to a mating piece;

FIG. 3: shows a longitudinal section of the arrangement from FIG. 2, but with a locked capping sleeve on the connecting piece;

FIG. 4: shows a top view of the capping sleeve and the connecting piece from FIG. 3 in the state in which the capping sleeve is locked to the connecting piece; and

FIG. 5: shows a cross section of the capping sleeve and the connecting piece from FIG. 3 in the state in which the capping sleeve is locked to the connecting piece.

### DETAILED DESCRIPTION

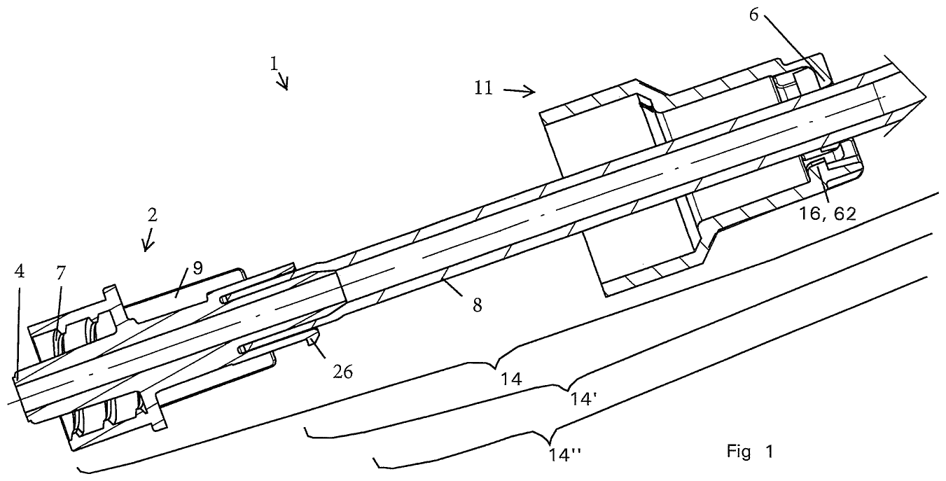

FIG. 1 shows a longitudinal section of a Luer lock connector 1 that has a connecting piece 2 according to the invention. A hose 8 is situated on this connecting piece 2 at a proximal end opposite from a Luer thread 7 situated in the connecting piece 2. In addition, the connecting piece 2 at the distal end has a Luer cone 4 that is at least partially enclosed by the Luer thread 7. It is apparent that in this embodiment the hose 8 is slipped onto a portion of the connecting piece 2 and is thereby stretched, and thus has a larger diameter than in its further course away from the connecting piece 2. However, this is not required. Outwardly pointing locking member 26 are mounted at the proximal end of the connecting piece.

Also shown is a capping sleeve 11 according to the invention which is arranged on the hose 8, normally by the manufacturer. This capping sleeve 11 is provided with locking member 62, which are to be understood as counterlocking member 62 with respect to the locking member 26 of the connecting piece 2.

Furthermore, the capping sleeve 11 has engagement mechanism 6. These engagement mechanism 6 hinder movement of the capping sleeve 11, situated on the hose 8, on a displacement area 14, wherein the displacement area 14 includes the area on which the capping sleeve 11 is displaceable relative to the connecting piece 2. The engagement mechanism 6 are configured in such a way that they form an opening width such that the engagement mechanism 6 rest on this displacement area 14 with an undersize, and exert a compressive force on the hose 8, so that a displacement of the capping sleeve 11 relative to the connecting piece 2 is possible only with the aid of additional force. Unintentionally moving and/or sliding the capping sleeve 11 are/is thus hindered or prevented. The weight force of the capping sleeve 11 by itself is not sufficient for the capping sleeve 11 to move on the displacement area 14.

Displacement on the stretched area of the hose 8 requires a further increase in the force, and ensures protection from inadvertent locking of the locking member 26 and 62.

The engagement mechanism 6 are situated distally with respect to the locking member 62 in FIG. 1, wherein the distance between the engagement mechanism 6 and the locking member 62 is configured in such a way that this distance creates a space within the capping sleeve that corresponds at least to the width of the locking member 26 of the connecting piece 2 parallel to the longitudinal extension of the connecting piece 2. The width of the locking member 26 illustrated in FIG. 1 corresponds to 1 mm, and the length of the space within the capping sleeve, defined by the distance between the engagement mechanism 6 and the locking member 62, is accordingly 1.5 mm.

In this regard, the locking member 26 of the connecting piece 2 have an advantageous design in the form of a trapezoidal shape, in that a first edge situated at the proximal end of the connecting piece 2 is broken off, which simplifies slipping on the capping sleeve 11 together with its locking member 62.

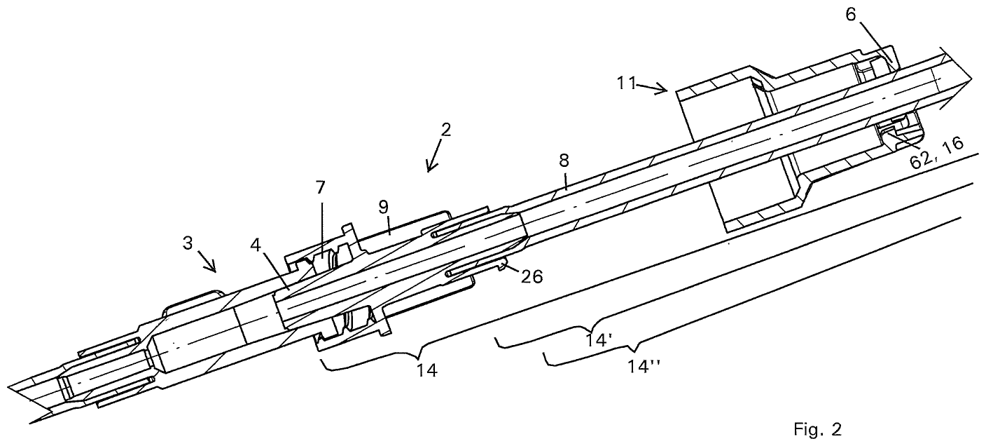

FIG. 2, in a longitudinal section of the connector 1 from FIG. 1, shows a mating piece 3 that fits the connecting piece 2, and a mating thread 17 that fits the thread 7, in the connected state, the connector being partially secured by screwing the thread 7 and the mating thread 17 together. The thread is not fully utilized in this illustration. The width of the insertion point of the cone 4 into the mating piece 3 varies greatly due to manufacturing tolerances, so that in practice, different numbers of threads are utilized for the securing. In addition, FIG. 2, the same as in FIG. 1, shows an advantageous spacer piece 9 which is provided in the connecting piece 2, and which via appropriate grip extensions simplifies screwing the connecting piece and a mating piece together. Also illustrated is the area 14′ on which the locking member 62 of the capping sleeve 11 can be moved, the locking member already being locked in the far right position in the figure via the locking member 26 of the connecting piece 2, and the area 14″ on which the engagement mechanism 6 of the capping sleeve can be moved, the locking member 62 already being locked in the far right position of the area 14″ of the engagement mechanism 6 in the figure via the locking member 26 of the connecting piece 2.

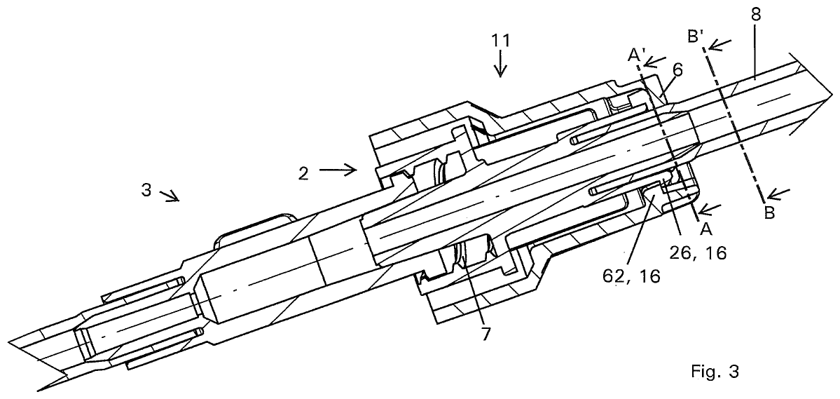

FIG. 3 illustrates a longitudinal section of the elements from FIG. 2, wherein the mating piece 3 and the connecting piece 2 are completely connected by further screwing and are secured by the screw connection. For this purpose, the capping sleeve 11 is locked to the connecting piece 2, and the arrangement is thus transferred into a nondisconnectable state.

In this state, the capping sleeve 11 is nonreleasably locked to the connecting piece 2 via the locking member 26 of the connecting piece 2 and the locking member 62 of the capping sleeve 11. In such a state, displacement of the capping sleeve 11 relative to the connecting piece 2 is greatly limited in all directions. The locking member 16 are configured in such a way that the capping sleeve 11 can freely rotate relative to the connecting piece 2, so that further influencing of the screw connection of the thread 7 with the mating thread 17 is not possible without further aids, or without destroying the capping sleeve 11.

The longitudinal extension of the capping sleeve 11 has been selected in such a way that in the state in which the capping sleeve 11 is locked to the connecting piece 2, the thread 7 of the connecting piece 2 and also the connecting piece 2 are at least covered in such a way that nondestructive access to the connecting piece 2 for loosening the screw connection is not possible without a tool. The area of the connecting piece 2 not covered by the capping sleeve 11 is so small that the screw connection cannot be loosened, at least by human hands.

On its section that accommodates the hose 8, the connecting piece 2 shown has an outer diameter that is larger than the inner diameter of the hose 8. The hose 8 is thus stretched at this section, as the result of which the hose 8, and thus the displacement area 14, at least at this section has an expanded outer diameter compared to the remainder of the hose 8. However, this is not required. In order to reach the state in which the capping sleeve 11 is locked to the connecting piece 2, the capping sleeve 11 on the displacement area 14 must be moved onto the connecting piece 2 with a greater force than is necessary for displacement on the remainder of the hose 8, since among other things, the engagement mechanism 6 must be moved onto a portion of the displacement area 14 having an expanded outer diameter. The larger force is greater than the force additionally required for moving the capping sleeve 11 onto the hose 8 of the displacement area 14.

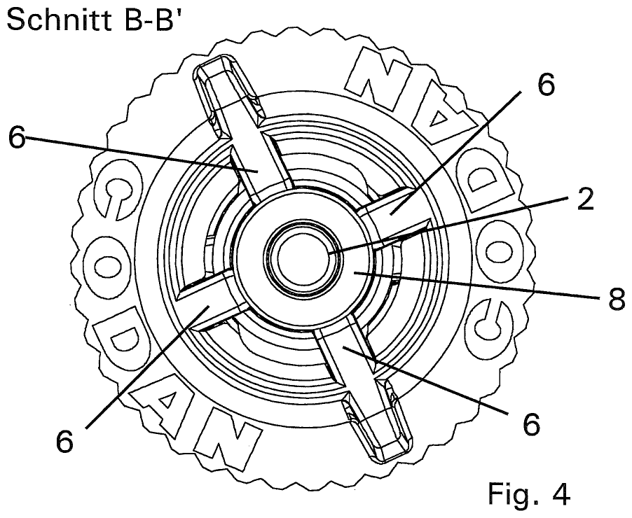

FIG. 4 illustrates a top view of the arrangement from FIG. 3 viewed from the line B-B′, showing the arrangement in a perspective looking onto the end of the capping sleeve 11, which at least in part does not cover the thread 7 of the connecting piece 2. Such a top view shows the engagement mechanism 6 of the capping sleeve 11 resting against the displacement area 14, in the present case, the hose 8. In this embodiment of the capping sleeve 11, four bars as engagement mechanism 6 are arranged on the capping sleeve 11, the bars being situated in such a way that ring segments form equal distances between the bars, so that the bars are arranged symmetrically in a circle around the displacement area 14. Due to the state in which the capping sleeve 11 is locked to the connecting piece 2, the engagement mechanism 6 rest on a portion of the hose 8, which has an enlarged outer diameter because of the accommodation of the hose 8 on the connecting piece 2.

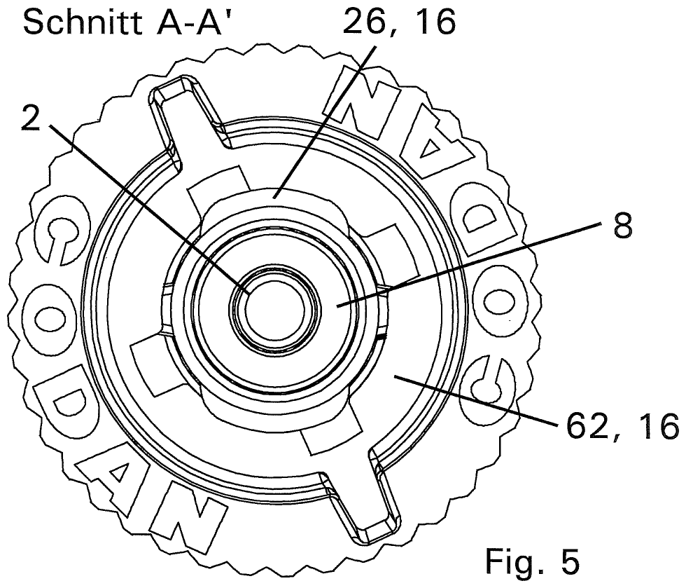

FIG. 5 illustrates a section at the level of the line A-A′ through the arrangement from FIG. 3, wherein the connector 1 is viewed starting from the end of the capping sleeve 11, which at least in part does not cover the thread 7 of the connecting piece 2.

The state in which the capping sleeve 11 is locked to the connecting piece 2 is shown, with the locking member 26 of the connecting piece 2 situated above the locking member 62. The outer diameter of the locking member 26 of the connecting piece 2 is larger than the diameter of the locking member opening width of the locking member 62 of the capping sleeve 11. This top view also shows that the locking member 26 of the connecting piece 2 together with the locking member 62 of the capping sleeve 11 result in almost a complete circle. Releasing by rotation is not possible. The connecting piece 2 is thus nonreleasably locked to the capping sleeve 11.

### LIST OF REFERENCE NUMERALS

1 connector
2 connecting piece
3 mating piece
4 cone
6 engagement mechanism
7 thread
8 hose
9 spacer piece
11 capping sleeve
14 displacement area
14′ area on which the locking member 62 of the capping sleeve can be displaced
14″ area on which the engagement mechanism 6 can be displaced
16 locking member
17 mating thread
26 locking member of the connecting piece
62 locking member of the capping sleeve

1. A connector and/or an adapter comprising:
a connecting piece adapted for connection to a fitting mating piece for a purpose of substance transport, wherein the connecting piece has:
a cone; and
a securing mechanism for reversibly securing a connection of the connecting piece to the fitting mating piece;
a hose;
a displacement area;
a capping sleeve configured to be at least partially rotatable about the connecting piece and/or on the displacement area; and
at least one locking member provided on the connecting piece and/or on the capping sleeve for nonreleasably locking the connecting piece to the capping sleeve;
wherein the connector is designed such that in a state in which the connecting piece is nonreleasably locked to the capping sleeve, displacement of the capping sleeve relative to the connecting piece is limited in all directions;
wherein the capping sleeve and the displacement area are designed such that the capping sleeve has at least one engagement mechanism configured to be at least partially connected to the displacement area and thus to at least partially enclose the displacement area such that:
(a) between the at least one engagement mechanism and the displacement area a resistance is present that acts against a displacement of the capping sleeve relative to the connecting piece, so that a force that is necessary to move the capping sleeve is greater than a weight force of the capping sleeve; and/or
(b) the at least one engagement mechanism forms an opening width which is selected so that the at least one engagement mechanism rests on the displacement area, exerts a force compressing the displacement area with an undersize on a circumference of the displacement area; and/or
(c) the at least one engagement mechanism defines an opening that is smaller than a maximum extension of a cross-section of the displacement area perpendicular to a longitudinal extension of the displacement area; and/or
(d) the capping sleeve is displaceable on the displacement area by means of a force of less than 5N and/or greater than 0.01N, wherein the force that displaces the capping sleeve on the displacement area acts parallel to the longitudinal extension of the displacement area and/or to the capping sleeve, in particular in addition to the weight force of the capping sleeve; and
the capping sleeve is designed such that in the state in which the connecting piece is nonreleasably locked to the capping sleeve:
the securing mechanism and all parts torsionally rigidly connected thereto are at least partially enclosed and/or covered, in particular by the capping sleeve; and/or
access to portions of the connector that are usable for releasing the securing mechanism and/or for reversing the securing, are at least partially or completely enclosed and/or covered, in particular by the capping sleeve; and/or
operating and/or releasing the securing mechanism is hindered and/or prevented by at least partially covering the securing mechanism and/or the fitting mating piece and/or the connector, in particular by covering with the capping sleeve.

2. The connector according to claim 1, wherein the capping sleeve is rotatable about the connector, at least in the state in which the capping sleeve is nonreleasably locked to the connecting piece.

3. The connector according to claim 1, wherein the at least one locking member is configured in such a way that the force required for the nonreleasable locking of the at least one locking member is at least 27.5N and/or at most 60N.

4. The connector according to claim 1, wherein in the nonreleasably locked state of the connector, the connector is releasable only by at least partially destroying the capping sleeve and/or the connector.

5. The connector according to claim 1, wherein the releasing of the reversible securing is hindered, in particular prevented, by the at least partial enclosure of the securing mechanism and/or of the connector and/or of the fitting mating piece brought about by the capping sleeve in the nonreleasably locked state.

6. The connector according to claim 1, wherein the opening width formed by the at least one engagement mechanism has a smaller diameter than a maximum outer diameter of the displacement area.

7. The connector according to claim 1, wherein the at least one locking member of the capping sleeve includes at least one first locking member that forms a locking member opening width, and wherein:
the at least one locking member of the connecting piece includes at least one second locking member that forms a locking member outer diameter, wherein a diameter of the locking member opening width is less than 8 mm and/or is smaller than the locking member outer diameter; and/or
the at least one second locking member of the connecting piece with its outer extensions defines a circle having an outer diameter that is smaller than an inner diameter of a portion of the capping sleeve, in and/or on which the at least one second locking member of the connecting piece is accommodated and/or rests in the state in which the connecting piece is nonreleasably locked to the capping sleeve.

8. The connector according to claim 1, wherein the at least one engagement mechanism has at least one bar.

9. The connector according to claim 1, wherein each of the at least one locking member is formed so as to have a trapezoidal shape in its cross-sectional plane parallel to the longitudinal extension of the connector and/or of the capping sleeve.

10. The connector according to claim 1, wherein the at least one locking member of the capping sleeve and/or of the connecting piece, in particular in the state in which the connecting piece is locked to the capping sleeve, form a ring that is almost completely closed.

11. The connector according to claim 1, wherein the at least one locking member of the connecting piece and/or of the capping sleeve, in a top view of the at least one locking member parallel to the longitudinal extension of the connecting piece and/or of the capping sleeve, forms a segment of a ring;
wherein the at least one locking member of the capping sleeve includes a first locking member and a second locking member; and wherein a longest chord through each segment of the at least one locking member of the connecting piece is longer than a distance between the first locking member and the second locking member of the capping sleeve, which the at least one locking member of the capping sleeve has in the top view of the at least one locking member parallel to the longitudinal extension of the connecting piece and/or of the capping sleeve; and/or
wherein the at least one locking member of the connecting pieces includes a first locking member and a second locking member; and wherein the at least one locking member of the connecting piece and/or of the capping sleeve, in the top view of the at least one locking member parallel to the longitudinal extension of the connecting piece and/or of the capping sleeve forms a segment of a ring, wherein the longest chord through each segment of the at least one locking member of the connecting piece is longer than the distance between the first locking member and the second locking member of the connecting piece, which the at least one locking member of the connecting piece has in the top view of the at least one locking member parallel to the longitudinal extension of the connecting piece and/or of the capping sleeve; and/or
the at least one of the locking member of the connecting piece and/or of the capping sleeve, in the top view of the at least one locking member parallel to the longitudinal extension of the connecting piece and/or of the capping sleeve, forms a segment of a ring, wherein a first locking member of the at least one locking member of the connecting piece is at least partially covered by a second locking member of the at least one locking member of the capping sleeve; and/or
the at least one locking member of the connecting piece and of the capping sleeve, in the top view of the at least one locking member parallel to the longitudinal extension of the connecting piece and/or of the capping sleeve form a segment of a ring, wherein in a state in which the connecting piece is locked to the capping sleeve, in any possible position of the at least one locking member of the connecting piece and of the capping sleeve, one of the at least one locking member of the connecting piece is at least partially covered by one of the at least one locking member of the capping sleeve.

12. The connector according to claim 1, wherein the at least one locking member of the connecting piece and/or of the capping sleeve, in a top view of the at least one locking member parallel to the longitudinal extension of the connecting piece and/or of the capping sleeve, form a segment of a ring;
wherein a longest chord through each segment of the at least one locking member of the connecting piece is longer than a distance between a first end and a second end of the at least one locking member of the capping sleeve, which the at least one locking member of the capping sleeve has in the top view of the at least one locking member parallel to the longitudinal extension of the connecting piece and/or of the capping sleeve.

13. The connector according to claim 1, wherein the securing mechanism is fixedly and/or rigidly connected to the cone and/or to the connecting piece.

14. The connector according to claim 1, wherein the at least one engagement mechanism of the capping sleeve is provided distally on the capping sleeve and/or on an end of the capping sleeve remote from the securing mechanism, and/or the capping sleeve is positioned in such a way that the at least one engagement mechanism of the capping sleeve is situated on an end of the connecting piece remote from the cone, and/or on the displacement area, on an end of the displacement area remote from the cone.

15. A system comprising at least one connector that has a connecting piece according to claim 1 and at least one fitting mating piece.

16. A method for producing an infusion arrangement for transport, said method comprising:
(i) reversibly connecting a connecting piece of a connector to a fitting mating piece, and wherein this connection is reversibly secured;
(ii) hindering or preventing by slipping on a capping sleeve of the connector, the releasing of the reversible securing; and nonreleasably locking the capping sleeve to the connecting piece; wherein the hindrance and/or prevention takes place by at least partially covering and/or at least partially enclosing the securing mechanism and/or the connecting piece and/or the fitting mating piece used for the reversible securing, and/or by hindering and/or preventing access to the securing mechanism used for the reversible securing, and/or by hindering and/or preventing operation and/or release of the securing mechanism used for the reversible securing;
(iii) wherein the slipping on and the nonreleasable locking is accomplished by moving the capping sleeve on the connecting piece and/or on a displacement area and/or on a hose and/or on a tube adjoining same, and/or by moving the capping sleeve on the connecting piece and/or by slipping the capping sleeve onto the connecting piece.

17. The method according to claim 16, further comprising:
providing the connection piece with a cone and a securing mechanism for reversibly securing a connection of the connecting piece to the fitting mating piece; and
providing the hose and the displacement area on the connector; and wherein the method further includes at least partially rotating the capping sleeve about the connecting piece and/or on the displacement area.

18. The method according to claim 17, further comprising:
providing at least one locking member on the connecting piece and/or on the capping sleeve; and
nonreleasably locking the connecting piece to the capping sleeve.

19. The method according to claim 16, further comprising:
providing a connector according to claim 1.

20. A method for producing an infusion arrangement for transport, with connection of a connecting piece to a fitting mating piece, comprising:
using a system that includes a connector comprising:
a connecting piece adapted for connection to a mechanism mating piece, wherein the connecting piece has a cone and a securing mechanism for reversibly securing a connection of the connecting piece to the fitting mating piece; a hose; a displacement area; a capping sleeve configured to be at least partially rotatable about the connecting piece and/or on the displacement area; and at least one locking member provided on the connecting piece and/or on the capping sleeve for nonreleasably locking the connecting piece to the capping sleeve;
reversibly connecting the connecting piece to the fitting mating piece, and wherein this connection is reversibly secured;
hindering or preventing by slipping on the capping sleeve, the releasing of the reversible securing; and nonreleasably locking the capping sleeve to the connecting piece; wherein the hindrance and/or prevention takes place by at least partially covering and/or at least partially enclosing the securing mechanism and/or the connecting piece and/or the fitting mating piece used for the reversible securing, and/or by hindering and/or preventing access to the securing mechanism used for the reversible securing, and/or by hindering and/or preventing operation and/or release of the securing mechanism used for the reversible securing;
wherein the slipping on and the nonreleasable locking is accomplished by moving the capping sleeve on the connecting piece and/or on a displacement area and/or on a hose and/or on a tube adjoining same, and/or by moving the capping sleeve on the connecting piece and/or by slipping the capping sleeve onto the connecting piece; and
wherein the connector is designed such that in a state in which the connecting piece is nonreleasably locked to the capping sleeve, displacement of the capping sleeve relative to the connecting piece is limited in all directions;
wherein the capping sleeve and the displacement area are further designed such that the capping sleeve has at least one engagement mechanism configured to be at least partially connected to the displacement area and thus to at least partially enclose the displacement area such that:
(a) between the at least one engagement mechanism and the displacement area a resistance is present that acts against a displacement of the capping sleeve relative to the connecting piece, so that a force that is necessary to move the capping sleeve is greater than a weight force of the capping sleeve; and/or
(b) the at least one engagement mechanism forms an opening width which is selected so that the at least one engagement mechanism rests on the displacement area, exerts a force compressing the displacement area with an undersize on a circumference of the displacement area; and/or
(c) the at least one engagement mechanism defines an opening that is smaller than a maximum extension of a cross-section of the displacement area perpendicular to a longitudinal extension of the displacement area; and/or
(d) the capping sleeve is displaceable on the displacement area by means of a force of less than 5N and/or greater than 0.01N, wherein the force that displaces the capping sleeve on the displacement area acts parallel to the longitudinal extension of the displacement area and/or to the capping sleeve, in particular in addition to the weight force of the capping sleeve; and
the capping sleeve is further designed such that in the state in which the connecting piece is nonreleasably locked to the capping sleeve:
the securing mechanism and all parts torsionally rigidly connected thereto are at least partially enclosed and/or covered, in particular by the capping sleeve; and/or
access to portions of the connector that are usable for releasing the securing mechanism and/or for reversing the securing, are at least partially or completely enclosed and/or covered, in particular by the capping sleeve; and/or
operating and/or releasing the securing mechanism is hindered and/or prevented by at least partially covering the securing mechanism and/or the fitting mating piece and/or the connector, in particular by covering with the capping sleeve.
